(19) United States
(12) Patent Application Publication
(10) Pub. No.: US 2024/0376325 A1
(43) Pub. Date: Nov. 14, 2024

(54) COATED SUBSTRATES THAT DEMONSTRATE SUPERHYDROPHILICITY, SUITABLE FOR USE AS MEDICAL DEVICES

(71) Applicant: ACULON, INC., San Diego, CA (US)

(72) Inventor: ERIC L. HANSON, Carlsbad, CA (US)

(21) Appl. No.: 18/668,499

(22) Filed: May 20, 2024

Related U.S. Application Data
(63) Continuation of application No. PCT/US2022/049827, filed on Nov. 15, 2022.
(60) Provisional application No. 63/281,280, filed on Nov. 19, 2021.
(60) Provisional application No. 63/281,016, filed on Nov. 18, 2021.

(51) Int. Cl. C09D 5/16; A61B 17/00; C09D 133/26; C09D 153/00

(52) U.S. Cl. CPC C09D 5/1668; C09D 133/26; C09D 153/00; A61B 2017/00942

(57) ABSTRACT

Coated articles and medical devices are provided comprising a substrate with a surface having reactive functional groups, a polymerization initiator chemically bonded to the substrate via reaction with the reactive functional groups on the surface of the substrate; and a polymeric coating layer having a thickness greater than 10 nanometers and less than 2.5 microns. The polymeric coating layer is prepared from an aqueous monomer composition comprising at least 50 percent by weight of at least one (meth)acrylamide monomer having at least one ionic functional group. The polymeric coating layer is chemically bonded to and propagated from the polymerization initiator, and the polymeric coating layer demonstrates a water contact angle less than 10 degrees. The coated article retains a contact angle of less than 10 degrees after immersion in phosphate buffered aqueous saline solution at 22° C. for a period of 28 days.

### RELATED APPLICATIONS

The present application is a continuation of International Patent Application Serial Number PCT/US2022/049827 filed Nov. 14, 2022 and published May 25, 2023, which application and publication are incorporated herein by reference in their entirety.

International Patent Application Serial Number PCT/US2022/049827 claims the benefit of provisional patent application Ser. No. 63/281,016, titled “Coated Substrates that Demonstrate Superhydrophilicity, Suitable for use as Medical Devices”, filed Nov. 18, 2021, and incorporated by reference in its entirety.

International Patent Application Serial Number PCT/US2022/049827 claims the benefit of provisional patent application Ser. No. 63/281,280, titled “Coated Substrates that Demonstrate Superhydrophilicity, Suitable for use as Medical Devices”, filed Nov. 19, 2021, and incorporated by reference in its entirety.

### FIELD OF THE INVENTION

The present invention relates to coated articles and specifically medical devices having thin coatings that demonstrate super-hydrophilicity and fouling resistance.

### BACKGROUND OF THE INVENTION

Changing the surface energy of a substrate is of great value in many diverse industries and products, including medical devices; oil, gas and energy production; and electronics. Depending on the needs and applications, substrate surfaces can be customized to exhibit hydrophilic, hydrophobic, oleophobic and oleophilic properties. Exceptionally hydrophilic surfaces are particularly desired in certain high humidity environments to prevent fogging, and in aqueous environments to minimize surface air bubbles. For example, lenses used in vivo are particularly subject to fogging, distorting images. Rendering the surface hydrophilic is helpful in reducing fogging and other detriments.

Scientifically, liquids with lower surface tensions (e.g., oil with surface tensions <30 mN/m) tend to wet solid surfaces (with specific surface energy) more than liquids with higher surface tensions (e.g., water with surface tension of 72.8 mN/m). Coated surfaces may be prepared to yield different contact angles for liquids with higher surface tensions compared to those with lower surface tensions.

The modification of substrate surfaces with super-hydrophilic coatings has been proposed; however, conventional coating processes typically generate films that are too thick for practical use in a medical device.

The modification of substrate surfaces with super-hydrophilic polymers has also been proposed, using controlled radical polymerization (CRP) processes to dictate polymer architecture. Polymerization of (meth)acrylamide monomers that may provide such extreme hydrophilicity has historically been difficult to achieve via controlled radical polymerization processes such as ATRP, likely due to the exceptionally high rate of polymerization of these monomers as well as their chemical interaction with the copper-based catalysts typically used for such polymerization processes.

In addition, running these polymerizations in water adds another significant challenge due to the fact that ATRP initiators are usually activated alkyl halide compounds, which are highly prone to hydrolysis and rapidly lose their ability to control polymerization. When these polymerizations are conducted on surfaces in an aqueous medium, the ratio of initiating groups to water is many orders of magnitude in favor of water, making hydrolysis reactions far more likely than propagation of a polymer from an initiating site. Hence, the reported attempts of ATRP of (meth)acrylamide polymers in water are scarce, and surface-propagated polymerizations of them are absent from the literature to our knowledge.

Modifying the surfaces of substrates, including some modification methods that use CRP processes, tend to have drawbacks that make them unsuitable for many substrates because these processes typically employ solvents that are too aggressive, leading to degradation and/or swelling of a polymer substrate, or corrosion of a metal substrate caused by such solvents. For example, the most commonly used strategies for binding a polymerization initiator to a polymer surface use aggressive chemical attack to create functional groups (like hydroxyl, amine, etc.), followed by exposure to polar aprotic solvents such as diethyl ether, dichloromethane, etc., containing the initiator (for example, alpha-bromoisobutryl bromide) and a base catalyst such as triethylamine.

These methodologies are too aggressive to be used on most polymers, excepting highly fluorinated or crosslinked aromatic polymers such as PTFE, PFA, polyimide and PEEK. Commonly used polymers such as polyamide, pebax, acrylics, polyesters, polyolefins, PVC, and the like, are rapidly degraded by the aforementioned processes.

It would be desirable to provide coated articles comprising commonly used substrates, useful as medical devices and demonstrating superhydrophilicity and anti-fouling.

### SUMMARY OF THE INVENTION

One aspect of the present invention provides coated articles and medical devices comprising:
(a) a substrate with a surface having reactive functional groups;
(b) a polymerization initiator chemically bonded to the substrate via reaction with the reactive functional groups on the surface of the substrate; and
(c) a polymeric coating layer having a thickness greater than 10 nanometers and less than 2.5 microns. The polymeric coating layer is prepared from an aqueous monomer composition comprising at least 50 percent by weight of at least one (meth)acrylamide monomer having at least one ionic functional group. The polymeric coating layer is chemically bonded to and propagated from the polymerization initiator, and the polymeric coating layer demonstrates a water contact angle less than 10 degrees. The coated article retains a contact angle of less than 10 degrees after immersion in phosphate buffered aqueous saline solution at 22° C. for a period of 28 days.

These and other advantages of the present invention are described in the following detailed description of the invention.

### DETAILED DESCRIPTION OF THE INVENTION

Other than in any operating examples, or where otherwise indicated, all numbers expressing quantities of ingredients, reaction conditions and so forth used in the specification and claims are to be understood as being modified in all instances by the term “about.” Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

As used in this specification and the appended claims, the articles “a,” “an,” and “the” include plural referents unless expressly and unequivocally limited to one referent.

The various aspects and examples of the present invention as presented herein are each understood to be non-limiting with respect to the scope of the invention.

As used in the following description and claims, the following terms have the meanings indicated below:

The terms “on”, “appended to”, “affixed to”, “bonded to”, “adhered to”, or terms of like import means that the designated item, e.g., a coating, film or layer, is either directly connected to (in contact with) the object surface, or indirectly connected to the object surface, e.g., through one or more other coatings, films or layers.

The coated articles and medical devices of the present invention comprise (a) a substrate with a surface having reactive functional groups. Substrates suitable for use in the preparation of the coated articles and medical devices of the present invention can include a metal selected from at least one of aluminum, copper, stainless steel, a metal oxide, nitinol, palladium, nickel, tantalum and titanium; or a polymer selected from at least one of a polyamide, a polyamide-polyether block copolymer, a poly(meth)acrylate, a polyester, a polyolefin, a polyisoprene, a polyurethane, a polyester-polyurethane copolymer, a polyimide, a cycloolefin polymer, a polyether ketone, a polysulfone, a polycarbonate and a polysiloxane.

The substrates may be porous or nonporous. Porous substrates may be inherently porous or may be perforated with ordered or random microarrays of microchannels. “Microchannels” are understood to be micro-dimensional fluidic channels (e.g., having average diameters on a micron or nanometer scale). In microtechnology, a microchannel is understood to have a hydraulic diameter below 1 millimeter.

The substrate (a) has reactive functional groups on the surface. Suitable functional groups include active hydrogen groups such as hydroxyl, amino, amido, thiol, carboxylic acid, and the like. The reactive functional groups allow for chemical bonding between the substrate (a) and a polymerization initiator.

The substrate may take any shape as desired for the intended application, such as flat, curved, bowl-shaped, tubular, or flexible freeform, depending on the final product. For example, the substrate may be in the form of a flat plate or a lens having two opposing surfaces. The thickness of the substrate likewise depends on the nature of the final product. The coating may be applied to one or all of the surfaces of the substrate, such as both of two opposing surfaces.

The substrate may comprise any of a number of different medical devices, industrial articles or components of articles, including medical diagnostic equipment, a needle, a syringe, a tube or pumping system used for biological media, a lens, an intraocular lens, an intraocular lens delivery system, a catheter, a breathing apparatus, an electronic device, an implantable device for humans, an electronic fluidic device, a sensor, a mold, a biological/DNA assay surface, filter media, a fluidic microchannel, a heat exchanger, oil spill remediation equipment or oil processing equipment.

Before bonding the polymerization initiator to the substrate, the surface of the substrate may be modified by any of a variety of well-known techniques such as corona or argon plasma discharge, or chemical etching (particularly using a NaOH or KOH solution), to generate the reactive functional groups, such as hydroxyl functional groups, on the substrate surface.

The coated articles and medical devices of the present invention further comprise (b) a polymerization initiator chemically bonded to the substrate via reaction with the reactive functional groups on the surface of the substrate. The polymerization initiator may be bonded to the substrate using conventional techniques, including physical vapor deposition (PVD) or chemical vapor deposition (CVD), to ensure a thin layer of molecular dimensions.

Any initiators known in the art for living polymerization processes are suitable, provided they may be chemically bonded to the substrate surface by reaction with the reactive functional groups. Organosilicon compounds may serve as an initiator. Suitable organosilicon compounds include alkoxysilane functional compounds such as (3-trimethoxysilyl)propyl-2-bromo-2-methylpropionate. Also suitable are organosilicon-containing compounds with ethylenically unsaturated groups, such as (3-trimethoxysilyl)propyl (meth)acrylate, and (3-trimethoxysilyl)propyl (meth)acrylamide. Also useful are organophosphorus acids chemically bonded to the substrate surface, wherein the organo portion of the organophosphorus acid contains an initiator moiety such as a halide group. Other halide-containing compounds including alkyl halide compounds, such as a-bromoisobutyryl bromide, are also suitable. Azo-initiators such as azobisbutyronitrile (AIBN), 1,1′-azobis(cyclohexanecarbonitrile), and 4,4-azobis (4-cyanopentanoic acid), and K₂S₂O₈, as used in addition-fragmentation chain transfer (RAFT) polymerization processes may also be employed.

The coated articles and medical devices of the present invention further comprise (c) a polymeric coating layer. The polymeric coating layer is chemically bonded to and propagated from the polymerization initiator (b). The polymeric coating layer is prepared from an aqueous monomer composition comprising at least 50 percent by weight, based on the total weight of monomers in the monomer composition, of at least one (meth)acrylamide monomer having at least one ionic functional group. For example, the polymeric coating layer (c) may be formed from an aqueous monomer composition comprising at least one of a (meth)acrylamide halide salt, 2-aminoethylmethacrylamide hydrochloride halide salt, N,N′-(3-(dimethylamino)propyl) methacrylamide, N,N-(3-dimethylamino)propyl)-methacryloylaminobutyl sulfonate, N,N-(3-dimethylamino)propyl)-methacryloylaminopropyl sulfonate, 2-acrylamidopropane-2-methyl-1-propane sulfonic acid salt, [3-(methacryloylamino)propyl]trimethylammonium chloride, [3-(acryloylamino)propyl]trimethylammonium chloride, N, N′-dimethyl (meth)acrylamide and salts thereof, and 3-[(3-(meth)acrylamidopropyl)dimethylammonio]propanoate. The polymeric coating layer (c) may comprise a homopolymer of any of the above monomers, or may comprise a copolymer, such as a block copolymer, of two or more of the above monomers.

The polymeric coating layer (c) is prepared via a controlled radical polymerization (CRP) or “living” process; i.e., a chain-growth polymerization that propagates with essentially no chain transfer and essentially no chain termination. The molecular weight of a polymer prepared by CRP can be controlled by the stoichiometry of the reactants, i.e., the initial concentration of monomer(s) and initiator(s). In addition, CRP also provides polymers having characteristics including, for example, narrow molecular weight distributions, e.g., PDI values less than 2.5, and well-defined polymer chain architecture, e.g., block copolymers and alternating copolymers. As used herein, the term “controlled radical polymerization” and related terms such as “controlled radical polymerization process” includes, but is not limited to, atom transfer radical polymerization (ATRP), single electron transfer polymerization (SETP), reversible addition-fragmentation chain transfer (RAFT), and nitroxide-mediated polymerization (NMP).

In forming the coated articles of the present invention, the surface of the substrate is first contacted with initiator molecules to bond the initiator to the substrate. The initiator-coated substrate is then contacted with the monomer composition described above and a CRP catalyst, and polymerized under CRP conditions in an aqueous medium to form a layer or film of (meth)acrylamide-containing polymer.

In an example using ATRP, the substrate surface having reactive functional groups is contacted with a compound containing in a terminal portion a functional group reactive with the reactive functional groups on the substrate surface, and in a second terminal portion, an initiator for ATRP. A self-assembled monolayer (SAM) is formed from the compound bonded to the substrate surface, with the initiator extending outwardly from the substrate surface. The SAM is contacted with an aqueous mixture comprising the monomer composition and an ATRP catalyst, and the monomer composition is polymerized to form a layer of (meth)acrylamide-containing polymer on the substrate surface, chemically bonded to and propagated from the polymerization initiator.

The ATRP polymerization catalyst is typically a transition metal compound, which participates in a reversible redox cycle with the initiator; and a ligand, which coordinates with the transition metal compound. The ATRP process is described in further detail in International Patent Publication No. WO 98/40415 and U.S. Pat. Nos. 5,807,937, 5,763,548 and 5,789,487 which are incorporated herein by reference.

Catalysts that may be used in the ATRP preparation include any transition metal compound. It is preferred that the transition metal compound not form direct carbon-metal bonds with the polymer chain. Transition metal catalysts useful in the present invention may be represented by the following general formula:

$${M}^{n +} {X}_{n}$$

wherein M is the transition metal, n is the formal charge on the transition metal having a value of from 0 to 7, and X is a counterion or covalently bonded component. Examples of the transition metal M include, but are not limited to, Cu, Fe, Au, Ag, Hg, Pd, Pt, Co, Mn, Ru, Mo, Nb and Zn. Examples of X include, but are not limited to, halide, hydroxy, oxygen, C₁-C₆ alkoxy, cyano, cyanato, thiocyanato and azido. A preferred transition metal is Cu (l) and X is preferably halide, e.g., chloride. Accordingly, a preferred class of transition metal catalyst is the copper halides, e.g., Cu (I) CI. It is also preferred that the transition metal catalyst contain a small amount, e.g., 1 mole percent, of a redox conjugate, for example, Cu (II) Cl₂, when Cu (I) CI is used. Additional catalyst useful in preparing the pigment dispersant are described in U.S. Pat. No. 5,807,937 at column 18, lines 29 through 56 which patent is incorporated herein by reference in its entirety. Redox conjugates are described in further detail in U.S. Pat. No. 5,807,937 at column 11, line 1 through column 13, line 38 which patent is incorporated herein by reference in its entirety.

Ligands that may be used in ATRP for preparation of the polymerization catalyst include compounds having one or more nitrogen, oxygen, phosphorus and/or sulfur atoms, which can coordinate to the transition metal catalyst compound, e.g., through sigma and/or pi bonds. Classes of useful ligands include tertiary aliphatic amines, unsubstituted and substituted pyridines and bipyridines; porphyrins; cryptands; crown ethers; e.g., 18-crown-6; polyamines, e.g., ethylenediamine; glycols, e.g., alkylene glycols, such as ethylene glycol; carbon monoxide; and coordinating monomers, e.g., styrene, acrylonitrile and hydroxyalkyl (meth)acrylates. Note that the phrase “and/or” when used in a list is meant to encompass alternative embodiments including each individual component in the list as well as any combination of components. For example, the list “A, B, and/or C” is meant to encompass seven separate embodiments that include A, or B, or C, or A+B, or A+C, or B+C, or A+B+C.

As used herein and in the claims, the term “(meth)acrylate” and similar terms refer to acrylates, methacrylates and mixtures of acrylates and methacrylates; similarly for (meth)acrylamide. A preferred class of ligands are the substituted bipyridines, e.g., 4,4′-dialkyl-bipyridyls. Additional ligands that may be used in preparing pigment dispersant are described in U.S. Pat. No. 5,807,937 at column 18, line 57 through column 21, line 43 which patent is incorporated herein by reference in its entirety.

The reducing agent may be any reducing agent capable of reducing the transition metal catalyst from a higher oxidation state to a lower oxidation state, thereby reforming the catalyst activator state. Such reducing agents include, for example, SO₂, sulfites, bisulfites, thiosulfites, mercaptans, hydroxylamines, hydrazine (N₂H₄), phenylhydrazine (Ph-NHNH₂), hydrazones, hydroquinone, food preservatives, flavonoids, beta carotene, vitamin A, α-tocopherols, vitamin E, propyl gallate, octyl gallate, BHA, BHT, propionic acids, ascorbic acid, sorbates, reducing sugars, sugars comprising an aldehyde group glucose, lactose, fructose, dextrose, potassium tartrate, nitriles, nitrites, dextrin, aldehydes, glycine, and transition metal salts. Water-soluble reducing agents are particularly suitable.

The above-mentioned ingredients are dissolved or suspended in an aqueous medium which may include in minor portions a diluent such as an organic solvent, for example, acetone or methanol. Also, solvents such as those containing oligo ethylene oxide and propylene oxide groups, such as diethylene glycol, diethylene glycol monomethyl ether and tripropylene glycol monomethyl ether may be used in minor portions. Such solvents may boost the activity of the catalyst. The concentration of the radically polymerizable monomers is typically from 5 to 70 percent by weight based on total weight of solution. The molar ratio of catalyst to monomer ranges from 1:5 to 1:500, such as 1:20 to 1:100; the molar ratio of ligand to catalyst ranges from 1:2 to 1:100, such as 1:2 to 1:5. The molar ratio of reducing agent to catalyst is from 1:0.1 to 10 such as 1:0.5 to 2.

The solution of the radically polymerizable monomer composition can be applied to the initiator-coated substrate by conventional means such as dipping, rolling, spraying, printing, stamping or wiping. The formation of the ATRP film or coating can occur at temperatures in the range of −10 to 150° C. and at pressures of 1-100 atmospheres, usually at ambient temperature and pressure. By “ambient” conditions is meant without the application of heat or other energy; for example, when a curable composition undergoes a thermosetting reaction without baking in an oven, use of forced air, irradiation, or the like to prompt the reaction, the reaction is said to occur under ambient conditions. Usually, ambient temperature ranges from 60 to 90° F. (15.6 to 32.2° C.), such as a typical room temperature, 72° F. (22.2° C.). The time for conducting the ATRP can vary depending on the thickness of the film desired. The polymeric coating layer (c) typically has a thickness greater than 50 nm and less than 2.5 microns, such as less than 2 microns, or less than 1 micron, or even less than 100 nm. Such thick coating layers of aqueous CRP-generated (meth)acrylamide polymer coating layers have not been achieved previously; the thickness of the coating contributes to lubricity. The thickness of the film can be monitored by Quartz Crystal Microgravometric (QCM) measurement and the time for the ATRP is typically from 30 to 600 minutes. After ATRP, the coated substrate is removed from any remaining solution by rinsing with a polar solvent and drying the coated substrate.

When the surface of the initiator-coated substrate is exposed to the aqueous solution of the radically polymerizable monomer composition and subjected to ATRP conditions, the monomers contained therein form covalent bonds with each other and with the initiator groups that are bonded to the surface of the substrate. As mentioned above, the resultant coating or film is relatively thick (compared to typical surface ATRP processes) with strong adhesion to the substrate. The resulting polymer has a low polydispersity index because chain transfer reactions are minimized. Lower polydispersity indices enable the molecular weight of the polymer to be controlled and optimized for the particular application intended.

The resultant coated articles are hydrophilic, even superhydrophilic, demonstrating lubricity making them useful for easy clean coatings, antifog coatings and mold release agents. The coating layer demonstrates a water contact angle less than 10°, typically less than 5°, and retains a contact angle of less than 10° after immersion in phosphate buffered (approximate pH of 7.4) aqueous saline solution at 22° C. for a period of 28 days, often greater than 28 days. Additionally, the coating layer will not lose its hydrophilic or lubricious properties under these conditions

By “superhydrophilicity” is meant a high degree of hydrophilicity, or attraction to water; in superhydrophilic materials, the contact angle of water is less than 10°, often less than 5°, even equal to 0°. The coatings are also antifouling and can be useful in applications such as coatings for ship hulls, implanted biomaterials, medical instruments and drug delivery apparatus. In some cases, the coated surface prevents adsorption of proteinaceous compounds (DNA, serum proteins, etc.)

Whereas particular embodiments of this invention have been described above for purposes of illustration, it will be evident to those skilled in the art that numerous variations of the details of the present invention may be made without departing from the scope of the invention as defined in the appended claims.

What is claimed is:

1. A coated article comprising:
(a) a substrate with a surface having reactive functional groups;
(b) a polymerization initiator chemically bonded to the substrate via reaction with the reactive functional groups on the surface of the substrate; and
(c) a polymeric coating layer having a thickness greater than 10 nanometers and less than 2.5 microns, wherein the polymeric coating layer is prepared from an aqueous monomer composition comprising at least 50 percent by weight, based on the total weight of monomers in the monomer composition, of at least one (meth)acrylamide monomer having at least one ionic functional group, and wherein the polymeric coating layer is chemically bonded to and propagated from the polymerization initiator (b); and
wherein the polymeric coating layer demonstrates a water contact angle less than 10°, and wherein said coated article retains a contact angle of less than 10° after immersion in phosphate buffered aqueous saline solution at 22° C. for a period of 28 days.

2. The coated article of claim 1, wherein the polymerization initiator (b) comprises alkyl halide functional groups.

3. The coated article of claim 1, wherein the substrate (a) comprises a metal selected from at least one of aluminum, copper, stainless steel, a metal oxide, nitinol, palladium, nickel, tantalum and titanium; or a polymer selected from at least one of a polyamide, a polyamide-polyether block copolymer, a poly(meth)acrylate, a polyester, a polyolefin, a polyisoprene, a polyurethane, a polyester-polyurethane copolymer, a polyimide, a cycloolefin polymer, a polyether ketone, a polysulfone, a polycarbonate and a polysiloxane.

4. The coated article of claim 3, wherein the substrate comprises reactive hydroxyl functional groups.

5. The coated article of claim 1, wherein the polymeric coating layer (c) has a thickness less than 2 microns.

6. The coated article of claim 1, wherein the polymeric coating layer (c) has a thickness less than 1 micron.

7. The coated article of claim 1, wherein the polymeric coating layer (c) has a thickness less than 100 nm.

8. The coated article of claim 1, wherein the polymeric coating layer (c) has a thickness greater than 50 nm.

9. The coated article of claim 1, wherein the polymeric coating layer demonstrates a water contact angle of less than 5 degrees.

10. The coated article of claim 1, wherein the substrate comprises medical diagnostic equipment, a needle, a syringe, a tube or pumping system used for biological media, a lens, an intraocular lens, an intraocular lens delivery system, a catheter, a breathing apparatus, an electronic device, an implantable device for humans, an electronic fluidic device, a sensor, a mold, a biological/DNA assay surface, filter media, a fluidic microchannel, a heat exchanger, oil spill remediation equipment or oil processing equipment.

11. A coated article comprising:
(a) a substrate with a surface having reactive functional groups;
(b) a polymerization initiator chemically bonded to the substrate via reaction with the reactive functional groups on the surface of the substrate; and
(c) a polymeric coating layer having a thickness greater than 10 nanometers and less than 2.5 microns, wherein the polymeric coating layer is prepared from an aqueous monomer composition comprising at least 50 percent by weight, based on the total weight of monomers in the monomer composition, of at least one (meth)acrylamide monomer having at least one ionic functional group, and wherein the polymeric coating layer is chemically bonded to and propagated from the polymerization initiator (b), wherein the polymeric coating layer is formed from an aqueous monomer composition comprising at least one of a (meth)acrylamide halide salt, 2-aminoethylmethacrylamide hydrochloride halide salt, N,N′-(3-(dimethylamino)propyl) methacrylamide, N,N-(3-dimethylamino)propyl)-methacryloylaminobutyl sulfonate, N,N-(3-dimethylamino)propyl)-methacryloylaminopropyl sulfonate, 2-acrylamidopropane-2-methyl-1-propane sulfonic acid salt, [3-(methacryloylamino)propyl]trimethylammonium chloride, [3-(acryloylamino)propyl]trimethylammonium chloride, N, N′-dimethyl (meth)acrylamide and salts thereof, and 3-[(3-(meth)acrylamidopropyl)dimethylammonio]propanoate; and
wherein the polymeric coating layer demonstrates a water contact angle less than 10°, and wherein said coated article retains a contact angle of less than 10° after immersion in phosphate buffered aqueous saline solution at 22° C. for a period of 28 days.

12. The coated article of claim 11, wherein the polymeric coating layer (c) comprises a homopolymer of a (meth)acrylamide halide salt, 2-hydrochloride halide salt, N,N′-(3-aminoethylmethacrylamide (dimethylamino)propyl) methacrylamide, N,N-(3-dimethylamino)propyl)-methacryloylaminobutyl sulfonate, N,N-(3-dimethylamino)propyl)-methacryloylaminopropyl sulfonate, 2-acrylamidopropane-2-methyl-1-propane sulfonic acid salt, [3-(methacryloylamino)propyl]trimethylammonium chloride, [3-(acryloylamino)propyl]trimethylammonium chloride, N, N′-dimethyl (meth)acrylamide and salts thereof, or 3-[(3-(meth)acrylamidopropyl)dimethylammonio]propanoate.

13. The coated article of claim 11, wherein the polymeric coating layer (c) comprises a block copolymer.

14. The coated article of claim 11, wherein the polymeric coating layer (c) is prepared via a controlled radical polymerization (CRP) process.

15. A medical device comprising:
(a) a substrate with a surface having reactive functional groups;
(b) a polymerization initiator chemically bonded to the substrate via reaction with the reactive functional groups on the surface of the substrate; and
(c) a polymeric coating layer having a thickness greater than 10 nanometers and less than 2.5 microns, wherein the polymeric coating layer is prepared from an aqueous monomer composition comprising at least 50 percent by weight, based on the total weight of monomers in the monomer composition, of at least one (meth)acrylamide monomer having at least one ionic functional group, and wherein the polymeric coating layer is chemically bonded to and propagated from the polymerization initiator (b); and wherein the polymeric coating layer demonstrates a water contact angle less than 10 degrees, and wherein said coated article retains a contact angle of less than 10 degrees after immersion in phosphate buffered aqueous saline solution at 22° C. for a period of 28 days.

16. The medical device of claim 15, wherein the substrate comprises medical diagnostic equipment, a needle, a syringe, a tube or pumping system used for biological media, a lens, an intraocular lens, an intraocular lens delivery system, a catheter, a breathing apparatus, an electronic device, an implantable device for humans, an electronic fluidic device, a sensor, a mold, a biological/DNA assay surface, filter media, or a microfluidic channel.

17. The medical device of claim 15, wherein the polymeric coating layer (c) has a thickness less than 1 micron.

18. The medical device of claim 15, wherein the polymeric coating layer demonstrates a water contact angle of less than 5 degrees.

19. The medical device of claim 15, wherein the substrate (a) comprises a metal selected from at least one of aluminum, copper, stainless steel, a metal oxide, nitinol, palladium, nickel, tantalum and titanium; or a polymer selected from at least one of a polyamide, a polyamide-polyether block copolymer, a poly(meth)acrylate, a polyester, a polyolefin, a polyisoprene, a polyurethane, a polyester-polyurethane copolymer, a polyimide, a cycloolefin polymer, a polyether ketone, a polysulfone, a polycarbonate and a polysiloxane.

20. The medical device of claim 19, wherein the polymeric coating layer (c) has a thickness less than 1 micron.
